(19) United States
(12) Patent Application Publication
(10) Pub. No.: US 2009/0156647 A1
(43) Pub. Date: Jun. 18, 2009

(54) Method for maintaining physiological pH levels during intensive physical exercise

(75) Inventors: Michele Molino, Mississauga (CA); Joseph MacDougall, Mississauga (CA)

Correspondence Address: TORYS LLP, 79 WELLINGTON STREET WEST, SUITE 3000, BOX 270, TD CENTRE, TORONTO, ON, M5K 1N2, CA

(73) Assignee: IOVATE T. & P. INC., Mississauga (CA)

(21) Appl. No.: 11/954,635

(22) Filed: Dec. 12, 2007

(51) Int. Cl. A61K 31/44; A61P 3/00

(52) U.S. Cl. 514/348

(57) ABSTRACT

A nutritional supplement comprising at least a therapeutically effective amount of pyridoxine α-hydroxyisocaproate is provided by the present invention. The ingredients of the present nutritional supplement act substantially simultaneously to maintain physiological blood and muscular pH and increase the time to muscular fatigue in a mammal during periods of repetitive forceful muscular exercise. A method of use is provided by the present disclosure.

### RELATED APPLICATIONS

This application is related to co-pending U.S. patent application Ser. No. ______, entitled “Preparations containing Pyridoxine and α-Hydroxyisocaproic acid (HICA)” filed on Dec. 12, 2007, the contents of which are hereby incorporated by reference in there entirety.

### FIELD OF THE INVENTION

The present invention relates to the method of use of a nutritional supplement for maintaining physiological blood and muscular pH and increasing the time to muscular fatigue in a mammal during periods of repetitive forceful muscular exercise. More specifically, the present invention relates to a method of use for a nutritional supplement comprising at least a salt of pyridoxine and α-hydroxyisocaproic acid (HICA).

### BACKGROUND OF THE INVENTION

It is commonly known that increased muscle mass, strength and extended muscular performance occur in the most effective manner when exercise routines are done to complete exhaustion. However, the problem arises that during these extended periods of exercise that metabolites from the breakdown of adenosine triphosphate (ATP), mainly hydrogen ions (H⁺) begin to accumulate leading to a decline in the pH levels of blood and muscle. The increase in acidity of the muscle, as a result of the accumulation of H⁺ ions, is directly linked to muscle fatigue, which ultimately causes a decrease in the duration of intensive bouts of exercise (Cooke R, Pate E. The effects of ADP and phosphate on the contraction of muscle fibers. Biophys J. November 1985;48(5):789-98). This fatigue is a result of the fact that the decreased intramuscular pH inhibits enzymes which are vital for energy production and the force-producing capacity of muscles (Febbraio M A, Dancey J. Skeletal muscle energy metabolism during prolonged, fatiguing exercise. J Appl Physiol. December 1999;87(6):2341-7).

The body has a number of mechanisms that act as intracellular buffering systems, including, amino acids, proteins, inorganic phosphate (Pᵢ), bicarbonate, creatine phosphate hydrolysis, and lactate production (Robergs R A, Ghiasvand F, Parker D. Biochemistry of exercise-induced metabolic acidosis. Am J Physiol Regul Integr Comp Physiol. 2004;287:R502-16), all of which act to bind or consume H⁺ to protect the cell against intracellular proton accumulation. However, during periods of intense exercise, these buffering systems are quickly overcome and an H⁺ accumulation results, leading to muscle damage and fatigue.

Additionally, during prolonged exercise the circulating levels of ammonia increase (Snow R J, Carey M F, Stathis C G, Febbraio M A, Hargreaves M. Effect of carbohydrate ingestion on ammonia metabolism during exercise in humans. J Appl Physiol. May 2000;88(5):1576-80), and muscle fatigue results. Increased levels of ammonia in muscle and plasma have been shown to be correlated to muscle exhaustion and muscle cramping (Brouns F, Beckers E, Wagenmakers A J, Saris W H. Ammonia accumulation during highly intensive long-lasting cycling: individual observations. Int J Sports Med. May 1990;11 Suppl 2;S78-84), during highly intensive endurance exercise.

In situations wherein extended periods of repetitive, forceful muscular contractions are desired, such as during exhaustive physical exercise, it would be advantageous for an individual to both decrease the levels of H⁺ and attenuate the accumulation of ammonia in muscle. In this regard, the duration of exercise before the onset of fatigue can be increased and muscle performance enhanced.

### SUMMARY OF THE INVENTION

The present invention is directed towards the method of use of a nutritional supplement, comprising at least a salt of pyridoxine and α-hydroxyisocaproic acid (HICA). The ingredients of the present nutritional supplement act substantially simultaneously to maintain physiological blood and muscular pH and increase the time to muscular fatigue in a mammal during periods of repetitive forceful muscular exercise.

### DETAILED DESCRIPTION OF THE INVENTION

In the following description, for the purposes of explanations, numerous specific details are set forth in order to provide a thorough understanding of the present invention. It will be apparent, however, to one of ordinary skill in the art that the present invention may be practiced without these specific details.

The present invention is directed towards the administration of a nutritional supplement comprising at least a salt of pyridoxine and α-hydroxyisocaproic acid (HICA) to an animal or human, wherein specific benefits are conferred by both the pyridoxine component and the HICA component. The preferred route of administration is oral. Disclosed in the description of the present invention is a use of pyridoxine α-hydroxyisocaproate in producing compositions for oral administration to provide benefits related to maintaining physiological blood and muscular pH and increasing the time to muscular fatigue in a mammal during periods of repetitive forceful muscular exercise. Furthermore, the present invention is particularly well suited for use in tablets, capsules and solutions.

In yet another aspect of the present invention, pyridoxine α-hydroxyisocaproate possesses a strong buffering action, owing to the base component, pyridoxine. The buffering action in the blood, as well as in the muscle, is important in order to mediate decreases in pH as a result of ATP utilization.

As used herein, the term “pyridoxine α-hydroxyisocaproate” is to be understood as the salt of pyridoxine with HICA reacted in an equimolar ratio.

As used herein, ‘pyridoxine’ refers to the chemical 2-methyl-3-hydroxy-4,5-dihydroxymethylpyridine, (CAS Registry No. 65-23-6), also known as 3-hydroxy-4,5-bis(hydroxymethyl)-2-methylpyridine, 3-hydroxy-4,5-dimethyl-α-picoline, 5-hydroxy-6-methyl-3,4-pyridinedimethanol, or Vitamin B6. Additionally, as used herein, ‘pyridoxine’ also includes derivatives of pyridoxine such as esters, and amides, and salts, as well as other derivatives, including derivatives having substantially similar pharmacoproperties to pyridoxine upon metabolism to an active form.

As used herein, ‘α-hydroxyisocaproic acid’ refers to the chemical 2-hydroxy-4-methylvaleric acid, (CAS Registry No. 498-36-2), also known as HICA, or leucic acid. Additionally, as used herein, ‘α-hydroxyisocaproic acid’ also includes derivatives of α-hydroxyisocaproic acid such as esters, and amides, and salts, as well as other derivatives, including derivatives having substantially similar pharmacoproperties to α-hydroxyisocaproic acid upon metabolism to an active form.

A used herein, the term ‘nutritional supplement’ includes dietary supplements, diet supplements, nutritional composition, supplemental dietary and other compositions similarly envisioned and termed not belonging to the conventional definition of pharmaceutical interventions as is known in the art. Furthermore, ‘nutritional compositions’ as disclosed herein belong to category of compositions having at least one physiological function when administered to a mammal by conventional routes of administration.

α-Hydroxyisocaproic acid (HICA)

α-Hydroxyisocaproic acid (HICA) is an end product of the metabolism of the branched-chain amino acid, Leucine. In human tissues, such as muscle and connective tissue; HICA occurs naturally. Foods which are produced by fermentation, such as some cheeses, may contain small amounts of HICA. HICA is a reduction product of the α-keto acid analog of Leucine, α-ketoisocaproic acid (KICA), and as such contributes to the free pools of branched-chain amino acids (BCAA). HICA belongs to the group collectively known as branched-chain amino acid analogs. Moreover, HICA, unlike KICA, is stable in solution and is better suited for oral administration, since it is absorbed via an active transporter in the human intestine (Friedrich M, Murer H, Sterchi E, Berger E G. Transport of L-leucine hydroxyl analogue and L-lactate in human small intestinal brush border membrane vesicles. Eur J Clin Invest. February 1992;22(2):73-8).

Branched-chain amino acid analogs like HICA and KICA are essentially nitrogen-free amino acids and may serve three roles in cases of nitrogen accumulation, 1) providing the dietary requirement for Leucine without increasing nitrogen intake; 2) reducing the amount of nitrogen that must be excreted from the body; and 3) increasing levels of Leucine, which plays a key role in protein turnover and prevents wasting of lean body mass. It is important to note that nitrogen accumulation can result from a number of situations, including the catabolism of proteins in muscle during exercise. Since branched-chain amino acid analogs may be reaminated back to their corresponding amino acid (e.g. HICA can be converted to KICA, which can subsequently be converted back to Leucine), they can act to provide the dietary requirements for BCAA without increasing level of ingested nitrogen (Boebek K P, Baker D H. Comparative utilization of the α-keto and D- and L-α-hydroxy analogs of Leucine, Isoleucine and Valine by chicks and rats. J Nutr. October 1982;112(10):1929-39). This reamination reaction will act to reduce ammonia accumulation in plasma and working cells, therefore resulting in diminished central and muscle fatigue and reduced occurrence of delayed onset muscular soreness (DOMS).

Administration of about 1.5 g of HICA daily after intense exercise for 42 days (Karila T, Seppala T. α-Hydroxyisocaproic acid (HICA)—a Leucine metabolite for muscle recovery following exercise. www.elmomed.com) resulted in a statistically significant increase in total lean soft tissue mass. Additionally it was noted that subjects receiving HICA experienced little to no DOMS. It is likely that this amelioration of DOMS is a result of inhibition of metalloproteinases, which are responsible for degradation of the extracellular matrix during tissue remodeling.

Additionally in high catabolic states, such as those induced by intensive exercise, both α-keto acids and α-hydroxy acid analogues of branched-chain amino acids may be oxidized for energy instead of the branched-chain amino acids themselves (Staten M A, Bier D M, Matthews D E. Regulation of valine metabolism in man: a stable isotope study. Am J Clin Nutr. December 1984;40(6):1224-34). Using the deaminated analogs (e.g. HICA) over the aminated forms (e.g. Leucine), will act to attenuate ammonia accumulation in working muscle thereby maintaining a favorable nitrogen balance in an individual following administration. Also, α-hydroxy acid analogues, like HICA, can be reaminated to yield the corresponding branched-chain amino acids (Hoffer L J, Taveroff A, Robitaille L, Mame O A, Reimer M L. Alpha-keto and alpha-hydroxy branched-chain acid interrelationships in normal humans. J Nutr. September 1993;123(9):1513-21). Thus, oral administration of HICA, which is actively taken up in the intestine, can act to increase levels of Leucine present in skeletal muscle, thus reducing the need for supplemental branched-chain amino acids which may detrimentally contribute to an increase unwanted blood and muscular nitrogen levels.

Furthermore, Leucine, produced by the reamination of HICA is able to stimulate protein synthesis as well as inhibit protein breakdown (Tischler M E, Desautels M, Goldberg A L. Does Leucine, leucyl-tRNA, or some metabolite of Leucine regulate protein synthesis and degradation in skeletal and cardiac muscle? J Biol Chem. Feb. 25, 1982;257(4):1613-21), both of which are favorable and desirable in working muscle as they result in increased skeletal muscle growth and decreased recovery time.

It is herein understood by the inventors that oral administration of a pyridoxine salt of HICA, namely pyridoxine α-hydroxyisocaproate, will act to increase muscular concentrations of Leucine by supplying a BCAA analogue which may be preferentially catabolized over Leucine for energy production and that is further reaminated to form Leucine. Additionally, it is also understood by the inventors that a pyridoxine salt of HICA, namely pyridoxine α-hydroxyisocaproate, will also act to decrease plasma ammonia levels and reduce DOMS following periods of intensive exercise, thus shortening the recovery time between exercise periods.

Pyridoxine

Pyridoxine is a pyridine ring that contains hydroxyl, methyl and hydroxymethyl substituents, and is converted by the body to its active form, pyridoxal 5-phosphate. While pyridoxine is often referred to as Vitamin B6 it is actually only one of three vitamers which make up Vitamin B6; the others being pyridoxal and pyridoxamine. The active form of pyridoxine in the body is pyridoxal 5-phosphate, which is a coenzyme for all transamination as well as some decarboxylation and deamination reactions.

Pyridoxal 5-phosphate is an important coenzyme involved in the decarboxylation of amino acids resulting in amines (Bender D A. Non-nutritional uses of Vitamin B6. Br J Nutr. January 1999;81(1):7-20). These amines include neurotransmitters such as γ-aminobutyrate, histamine, noradrenaline, and serotonin.

Additionally, pyridoxal 5-phosphate is required as a coenzyme for all transamination reactions that occur in the body (Peterson D L, Martinez-Carrion M. The mechanism of transamination. Function of the histidyl residue at the active site of supernatant aspartate transaminase. J Biol Chem. Feb. 25, 1970;245(4):806-13). A transamination is the transfer of the amino group from an amino acid to an a-keto acid, e.g. α-ketoisocaproic acid can be converted to Leucine in this manner. As the product of transamination reactions depend on the availability of α-keto acids, providing exogenous HICA, which can be converted into KICA, would make the formation of Leucine more favorable.

Pyridoxal 5-phosphate gains its versatility for use in various metabolic pathways, since it is able to form a Schiff base between its aldehyde group and the amino group of an α-amino acid (Murray R K, Granner D K, Mayes P A, Rodwell V W. Harper's Biochemistry. Twenty-fifth edition. 2000. pg. 633. Appleton & Lange. Stamford, Conn.). This Schiff base formation allows the pyridoxal 5-phosphate to facilitate changes in the three remaining bonds of the amino carbon in order to allow transaminase, decarboxylation or threonine aldose activity.

It is herein understood by the inventors that oral administration of pyridoxine, provided as pyridoxine α-hydroxyisocaproate, will act to increase the conversion of HICA to Leucine in muscle, resulting in a lowering of plasma and cellular ammonia.

As used herein, a serving of the present nutritional supplement comprises from about 0.002 g to about 0.2 g of pyridoxine α-hydroxyisocaproate salt. More preferably, a serving of the present nutritional supplement comprises from about 0.050 g to about 0.175 g of pyridoxine α-hydroxyisocaproate salt. A serving of the present nutritional composition most preferably comprises from about 0.1 g to about 0.15 g of pyridoxine α-hydroxyisocaproate salt. Additionally, α-hydroxyisocaproic acid, in the non-salt form, may be present in the nutritional supplement.

Pyridoxine α-hydroxyisocaproate is used advantageously alone or with additional active ingredients, such as, trace elements, other vitamins, mineral substances, or other amino acids as well as, optionally, excipients usually used for the preparation of the respective forms of administration. The forms of administration include, particularly, all varieties of tablets, both those that are swallowed without being chewed, and tablets to be chewed or dissolved in the mouth of an individual, as well as those that are dissolved in a liquid before being ingested by an individual. The tablet forms include uncoated tablets, one-layer or multilayer or encased form or effervescent tablets. Further preferred forms of administration are capsules of hard and soft gelatin, the latter particularly suitable to include a liquid core. Additionally, pyridoxine α-hydroxyisocaproate can be used advantageously for the preparation of solutions and suspensions and as a powder, either effervescent or granulated.

Embodiments of the present invention having multi-phasic release profiles may produce physiologically relevant effects according the methods disclosed in U.S. patent application Ser. No. 11/709,525 entitled “Method for a Supplemental Dietary Composition Having a Multi-Phase Dissolution Profile” filed Feb. 21, 2007, which is herein fully incorporated by reference. The aforementioned discloses a method of providing a multi-phasic dissolution profile through the use of differentially-sized milled particles.

While not wishing to be bound by theory, it is understood by the inventors that pyridoxine α-hydroxyisocaproate and its derivatives are useful compounds, since they combine within a single molecule both the pyridoxine and the α-hydroxyisocaproate, thus resulting in the increase of the useful activities of these two compounds. Particularly, it is herein understood by the inventors that pyridoxine α-hydroxyisocaproate will have enhanced pH stability in water within a substantially broad range of concentrations.

Additionally, it is herein understood by the inventors that the pyridoxine component of the salt will act to increase the transamination of amino acids in muscle. This transamination acts to facilitate the conversion of HICA to Leucine, thereby increasing the levels of Leucine in the muscle. Greater conversion of HICA to Leucine will also act to decrease ammonia accumulation in plasma and muscle, thereby retarding the onset of central and muscle fatigue and reducing DOMS following intensive periods of exercise, thus shortening the recovery time between exercise periods.

Further to the aforementioned functions, it is also understood by the inventors that the a-hydroxyisocaproate component of the salt will act to increase muscular concentrations of Leucine by supplying a BCAA analogue that may be preferentially catabolized over that of Leucine to produce energy and is reaminated to form Leucine. Furthermore, it is herein understood by the inventors that the components of the present invention will act in concert through at least the aforementioned distinct mechanisms to reduce muscular fatigue following intensive exercise and to attenuate DOMS.

Additional embodiments of the present invention may also include portions of the composition as fine-milled ingredients. U.S. patent application Ser. No. 11/709,526 entitled “Method for Increasing the Rate and Consistency of Bioavailability of Supplemental Dietary Ingredients” filed Feb. 21, 2007, which is herein fully incorporated by reference, discloses a method of increasing the rate of bioavailability following oral administration of components comprising supplemental dietary compositions by the process of particle-milling.

According to various embodiments of the disclosure, pyridoxine α-hydroxyisocaproate may be used advantageously alone or with additional active ingredients to form a nutritional composition that may be consumed in any form. For instance, the dosage form of the nutritional composition may be provided as, e.g. a powder beverage mix, a liquid beverage, a ready-to-eat bar or ready-to-drink beverage product, a capsule, a liquid capsule, a tablet, a caplet, or as a dietary gel. The preferred dosage forms of the present invention are provided as a caplet or as a liquid capsule.

Furthermore, the dosage form of the nutritional composition may be provided in accordance with customary processing techniques for herbal and nutritional compositions in any of the forms mentioned above. Additionally, the nutritional composition set forth in the example embodiment herein disclosed may contain any appropriate number and type of excipients, as is well known in the art.

Although the following examples illustrate the practice of the present invention in three of its embodiments, the examples should not be construed as limiting the scope of the invention. Other embodiments will be apparent to one of skill in the art from consideration of the specifications and example.

### EXAMPLES

### Example 1

A nutritional supplement comprising the following ingredients per serving is prepared for consumption as a caplet to be administered once daily, preferably before meals:

About 0.150 g of pyridoxine α-hydroxyisocaproate salt.

### Example 2

A nutritional supplement comprising the following ingredients per serving is prepared for consumption as a caplet to be administered once daily, preferably before meals:

About 0.150 g of pyridoxine α-hydroxyisocaproate salt, and about 0.500 g of α-hydroxyisocaproic acid (non-salt form).

### Example 3

A nutritional supplement comprising the following ingredients per serving is prepared for consumption as a powder to be administered before engaging in physical exercise:

About 0.150 g of pyridoxine α-hydroxyisocaproate salt, about 7.20 g of Leucine, about 2.90 g of Creatine monohydrate, about 0.020 g of Creatine taurinate, about 0.080 g of Creatine malate, about 0.050 g of Coleus forskohlii extract, and about 17.50 g of dextrose monohydrate.

### Extensions and Alternatives

In the foregoing specification, the invention has been described with a specific embodiment thereof; however, it will be evident that various modifications and changes may be made thereto without departing from the broader spirit and scope of the invention.

What is claimed:

1. A method for attenuating metabolic acidosis and decreasing ammonia accumulation in blood and muscle comprising the step of administering to a mammal a composition comprising an effective amount of pyridoxine α-hydroxyisocaproate.

2. A method of claim 1, wherein the attenuation of metabolic acidosis and the decrease in ammonia accumulation in blood and muscle act to reduce central and muscular fatigue and diminish the symptoms of delayed onset muscle soreness (DOMS).

3. The method of claim 1, wherein at least a portion of the pyridoxine α-hydroxyisocaproate is fine-milled.

4. The method of claim 1, wherein the pyridoxine α-hydroxyisocaproate is provided as solid oral dosage form having a multi-phasic rate of dissolution.

5. The method of claim 4, wherein said multi-phasic rate of dissolution comprises a first-phase and a second-phase; whereby said first-phase has a first rate of dissolution and said second-phase has a second rate of dissolution.

6. The method of claim 5, further comprising a third-phase, whereby said third-phase has a third rate of dissolution.
